(19) United States
(12) Patent Application Publication
(10) Pub. No.: US 2008/0113421 A1
(43) Pub. Date: May 15, 2008

(54) Stabilizing proteins against denaturation and inactivation by charged detergents using chemical modifications, including modifications that increase net charge

(75) Inventors: Bryan F. Shaw, Somerville, MA (US); Katherine L. Gudiksen, San Francisco, CA (US); Irina Gitlin, Brookline, MA (US); Demetri T. Moustakas, Medford, MA (US); George M. Whitesides, Newton, MA (US)

Correspondence Address: WOLF GREENFIELD & SACKS, P.C., 600 ATLANTIC AVENUE, BOSTON, MA, 02210-2206, US

(73) Assignee: President and Fellows of Harvard College, Cambridge, MA (US)

(21) Appl. No.: 11/637,203

(22) Filed: Dec. 11, 2006

Related U.S. Application Data
(60) Provisional application No. 60/858,828, filed on Nov. 14, 2006.

(51) Int. Cl. C12N 9/96

(52) U.S. Cl. 435/188

(57) ABSTRACT

The present invention generally relates to enzymes and other proteins resistant to denaturation, and techniques for making and using the same. In one aspect, lysine and/or other charged residues within an enzyme are reacted in some fashion, which can render the enzyme more resistant to denaturation. For example, the lysine residue may be neutralized by acetylating the residue, for instance, by exposure to acetic anhydride. In some aspects, the enzyme, after reaction, may be relatively resistant to degradation when placed in a harsh environment, for example, when exposed to sodium dodecyl sulfate at a concentration of at least about 2.5 mM in Tris-Gly buffer. The enzyme may still be susceptible to denaturation in some cases, but at a much slower rate (e.g., the denaturation time constant may be higher). Other aspects of the invention are directed to enzymes prepared in such fashion, methods of promoting or using such enzymes, kits involving such enzymes, and the like.

### RELATED APPLICATIONS

This application claims the benefit of U.S. Provisional Patent Application Ser. No. 60/858,828, filed Nov. 14, 2006, by Shaw, et al., incorporated herein by reference.

### FIELD OF INVENTION

The present invention generally relates to proteins and, in particular, to proteins and enzymes resistant to denaturation, and techniques for making and using the same.

### BACKGROUND

Enzymes are biological proteins that catalyze chemical reactions of substrates into products. In general, enzymes are very selective and show a high degree of specific activity for their substrates. Because of this specificity, they have found increasing use in household and/or industrial uses, for instance, in applications such as laundry detergent, dishwashing detergent, contact lens cleaner, carpet cleaning solutions, and pet stain removers. However, enzymes within such environments often become denatured (e.g., losing their native conformation and thereby becoming inactive or non-catalytic), as such environments are typically unlike those encountered in nature where enzymes originate (e.g., within cells). As such, improvements in enzyme stability are needed.

### SUMMARY OF THE INVENTION

This invention generally relates to proteins resistant to denaturation, and techniques for making and using the same. The subject matter of the present invention involves, in some cases, interrelated products, alternative solutions to a particular problem, and/or a plurality of different uses of one or more systems and/or articles.

One aspect of the invention is directed to an article. In one set of embodiments, the article includes an enzyme or other protein, having a plurality of lysine and/or other charged residues, in which at least about 50% of the lysine and/or other charged resides of the enzyme or other protein have been acetylated.

The article, in another set of embodiments, includes an acetylated enzyme or other protein having a specific activity, with respect to the substrate of the enzyme or other protein, of at least about 75% relative to an unacetylated form of the enzyme or other protein.

In yet another set of embodiments, the article includes an acetylated enzyme or other protein that, when exposed to sodium dodecyl sulfate at a concentration of at least about 2.5 mM in Tris-Gly buffer, denatures with a time constant of less than about 175 h.

In one set of embodiments, the article includes an enzyme having a plurality of lysine and/or other charged residues reacted with an anhydride, the enzyme having a specific activity, with respect to the substrate of the enzyme, of at least about 75% relative to the enzyme when none of the lysine and/or other charged residues have been reacted. The article, in another set of embodiments, includes an enzyme or other protein that, when exposed to sodium dodecyl sulfate at a concentration of at least about 2.5 mM in Tris-Gly buffer, denatures with a time constant of less than about 175 h.

Another aspect of the invention is directed to a method. The method, in one set of embodiments, includes acts of providing an enzyme able to catalyze a substrate, and reacting a lysine and/or other charged residue of the enzyme with an acetylating agent to produce an acetylated enzyme such that the acetylated enzyme retains a specific activity, with respect to the substrate of the enzyme, of at least about 75% relative to the enzyme prior to the reacting step. In another set of embodiments, the method includes an act of reacting a lysine and/or other charged residue of a protein with an acetylating agent.

In still another set of embodiments, the method includes acts of providing an enzyme able to catalyze a substrate, and reacting a lysine and/or other charged residue of the enzyme with an acetylating agent to produce an acetylated enzyme such that the acetylated enzyme, when exposed to sodium dodecyl sulfate at a concentration of at least about 2.5 mM in Tris-Gly buffer, denatures with a time constant of less than about 175 h.

The method, in one set of embodiments, includes acts of providing an enzyme able to catalyze a substrate, and exposing the enzyme to an anhydride such that the anhydride reacts with at least one lysine and/or other charged residue on the enzyme such that the enzyme retains a specific activity, with respect to the substrate of the enzyme, of at least about 75% relative to the enzyme prior to reaction. In another set of embodiments, the method includes an act of neutralizing a lysine and/or other charged residue on a protein by exposing the protein to an anhydride.

In yet another set of embodiments, the method includes acts of providing an enzyme able to catalyze a substrate, and exposing the enzyme to an anhydride such that the anhydride reacts with at least one lysine and/or other charged residue on the enzyme such that the enzyme, when exposed to sodium dodecyl sulfate at a concentration of at least about 2.5 mM in Tris-Gly buffer, denatures with a time constant of less than about 175 h.

Still another aspect of the invention is directed to a method of promotion. In one set of embodiments, the method includes a method of promoting use of an acetylated enzyme or other protein in a detergent. In another set of embodiments, the method includes a method of promoting use, in a detergent, of an enzyme or other protein in which a plurality of lysine and/or other charged residues within the enzyme have been reacted with an anhydride.

Yet another aspect of the invention contemplates a kit. The kit, according to one set of embodiments, includes an acetylating agent, and instructions for reacting the acetylating agent with an enzyme or other protein. In another set of embodiments, the kit includes an agent able to react with a lysine and/or other charged residue, and instructions for reacting the agent with an enzyme or other protein.

In another aspect, the present invention is directed to a method of making one or more of the embodiments described herein. In another aspect, the present invention is directed to a method of using one or more of the embodiments described herein.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1B illustrate unfolding of BCA and BCA-Ac₁₈, in accordance with one embodiment of the invention;

FIGS. 2A-2B illustrate refolding of BCA and BCA-Ac₁₈, in accordance with another embodiment of the invention;

FIG. 3 illustrates various rate constants for unfolding and refolding of BCA and BCA-Ac₁₈, in accordance with yet another embodiment of the invention;

FIGS. 4A-4B illustrate refolding of BCA and BCA-Ac₁₈ after denaturation in SDS, in still another embodiment of the invention;

FIG. 5 is a schematic representation of various factors involved with protein denaturation, in one embodiment of the invention;

FIGS. 6A-6B show denaturation of hydrophobic charge ladders, according to another embodiment of the invention;

FIGS. 7A-7B show absorbance as a function of time for certain hydrophobic charge ladders, according to yet another embodiment of the invention;

FIGS. 8A-8B show rate constants of denaturation, in still another embodiment of the invention; and

FIGS. 9A-9B show various energy contributions, in yet another embodiment of the invention.

### DETAILED DESCRIPTION

The present invention generally relates to enzymes and other proteins resistant to denaturation, and techniques for making and using the same. In one aspect, lysine and/or other charged residues within an enzyme are reacted in some fashion, which can render the enzyme more resistant to denaturation. For example, the lysine residue may be neutralized by acetylating the residue, for instance, by exposure to acetic anhydride. In some aspects, the enzyme, after reaction, may be relatively resistant to degradation when placed in a harsh environment, for example, when exposed to sodium dodecyl sulfate at a concentration of at least about 2.5 mM in Tris-Gly buffer. The enzyme may still be susceptible to denaturation in some cases, but at a much slower rate (e.g., the denaturation time constant may be higher). Other aspects of the invention are directed to enzymes prepared in such fashion, methods of promoting or using such enzymes, kits involving such enzymes, and the like.

One aspect of the invention is directed to enzymes and other proteins that have been stabilized against denaturation. In one set of embodiments, charged residues, such as lysine, aspartate, glutamate, histidine, and/or arginine, on the enzyme or other protein are stabilized in some fashion, for instance, by neutralizing the residues (i.e., with respect to charge), thereby enhancing the stability of the enzyme or other protein during exposure to a surfactant, such as an anionic (e.g., a linear alkyl benzene sulfonate), neutral, or a cationic surfactant. Any agent able to neutralize the charged residue may be used. The charged residue may be neutralized, according to some embodiments, by reacting the residue with an agent that alters the chemical structure of the residue, for instance, such that it cannot readily form a charged state. For instance, the residue may be acetylated (i.e., an acetyl group is added to the residue) with a suitable agent, thereby stabilizing the enzyme or other protein against denaturation. Other suitable agents are described in detail below. The agent may be selected such that reaction of the agent with the enzyme or other protein does not substantially reduce the activity of the enzyme, or results in a minimal or acceptable loss of activity, as discussed in detail below.

As a non-limiting example of such an agent, a lysine residue may be neutralized by reacting the residue with an acetylating agent, i.e., an agent that, when reacted with the residue, adds an acetyl group to the residue. Acetylation of the residues, in many cases, does not substantially reduce the activity of the enzyme or other protein. Non-limiting examples of acetylating agents include acetic anhydride, acetyl chloride, acetyl bromide, succinimidyl ester, or methyl thioester. Thus, a residue having a charged amine (e.g., —NH₃⁺), upon reaction with an acetylating agent, may form a —NH—CO—CH₃ residue, which cannot be readily charged. Without wishing to be bound to any theory, it is believed that the absence of charged residues, such as lysine, causes slower denaturation of the enzyme when the enzyme is exposed to a surfactant (which is typically charged), due to a lack (or at least a reduction) of charge interactions with the surfactant.

Acetylation of the protein or other enzyme may be partial or total. For instance, at least about 50%, at least about 60%, at least about 75%, at least about 80%, at least about 85%, at least about 90%, or at least about 95% of the charged resides of the enzyme may be acetylated. For instance, if an enzyme has multiple lysine (or other charged) residues (e.g., 5 or more, 10 or more, 15 or more, 20 or more, etc.), then some or all of the residues may be reacted. In some cases, partially acetylation of the enzyme or other protein may be useful to simplify the reaction, allowing monitoring of the reaction (e.g., through capillary electrophoresis, mass spectroscopy, and/or other techniques for monitoring enzyme or protein reactions known to those of ordinary skill in the art), and/or preventing denaturation or distortion of the enzyme or other protein, which may reduce activity. As a non-limiting example, a molar equivalent of an acetylating reagent may be added to the enzyme or other protein.

In some embodiments of the invention, the enzyme may be stabilized against denaturation by reacting one or more charged residues of the enzyme with an anhydride, such as acetic anhydride, hexanoic anhydride, propionic anhydride, butyric anhydride, etc. Any anhydride able to react with a charged residue may be used. In some cases, the anhydride may react with one or more amine residues on the enzyme or other protein to form an —NH—CO—R moiety, e.g.:

—NH₃⁺+R—C(O)—O—C(O)—R→—NH—CO—R.

The reaction with the anhydride may be partial or total. For instance, at least about 50%, at least about 60%, at least about 75%, at least about 80%, at least about 85%, at least about 90%, or at least about 95% of the charged resides of the enzyme may be reacted. For instance, if an enzyme has multiple lysine residues (e.g., 5 or more, 10 or more, etc.), then some or all of the residues may be reacted.

A non-limiting example of an enzyme which may be used in the above-described reactions is bovine carbonic anhydrase II (BCA II). More generally, virtually any negatively charged enzyme can be stabilized from denaturation, for instance in an anionic detergent. In some embodiments, any protein in a pH environment higher than the isoelectric point (pI) of the protein (pH>pI) may be stabilized using the systems and methods discussed herein, and those of ordinary skill in the art will be able to determine the pI of a protein using no more than routine experimentation. Non-limiting examples of enzymes that may be stabilized at pHs between about 9 and about 11 include a variety of proteases, amylases, cellulases, lipases, or catalases.

Enzyme stability may be measured, in one set of embodiments, by comparing the specific activity of the enzyme after reaction with the unaltered enzyme (e.g., prior to acetylation or other reaction, as described above). Typically, the enzyme, after acetylation or other reaction, retains a specific activity towards its substrate of at least about 75%, and in some cases, at least about 80%, at least about 85%, at least about 90%, or at least about 95%. Those of ordinary skill in the art will be aware of systems and methods for measuring the specific activity of an enzyme with its substrate.

In another set of embodiments, the stability of the enzyme may be determined by exposing the enzyme to a detergent, and measuring the time constant of denaturation of the enzyme. A non-limiting example of a suitable detergent is sodium dodecyl sulfate at a concentration of at least about 2.5 mM in Tris-Gly buffer (25 mM Tris, 192 mM glycine, pH 8.4 at 25° C.). An enzyme that has been rendered resistant to denaturation typically will have a relatively high time constant, for example, at least about 50 h, at least about 75 h, at least about 100 h, at least about 125 h, at least about 150 h, at least about 175 h, at least about 200 h, etc. In some cases, the stability of the enzyme may be determined relative to the unaltered enzyme (e.g., prior to acetylation, as described above). For instance, the time constant of denaturation may be increased by a factor of at least about 2, at least about 5, at least about 10, at least about 30, at least about 100, at least about 300, at least about 1000, at least about 3000, at least about 10,000, at least about 30,000, or more in some cases.

The above-described compositions and methods also finds use in a wide variety of household and/or industrial uses in which one or more enzymes are used, for instance, in applications such as laundry detergent, dishwashing detergent, contact lens cleaner, carpet cleaning solutions, and pet stain removers, according to another aspect of the invention. Non-limiting examples of laundry detergents containing enzymes include Tide, Snow, Dreft, Cheer, Era, Ace, Bold, Gain (Proctor and Gamble); Whisk (Lever); Liquid Laundry Detergent (The Seventh Generation); Spray n' Wash, Spray n' Wash Dual Power Laundry Stain Remover, Spray n' Wash Stain Stick (Reckitt Benckiser, Inc.), or Arm and Hammer Fabricare (Church & Dwight Co., Inc.). Non-limiting examples of dish detergent containing enzymes include Cascade (Proctor and Gamble). Non-limiting examples of carpet cleaners containing enzymes include DooBeGone, Simple Solution Carpet Shampoo. Non-limiting examples of contact lens cleaner include Renu Multi-Purpose Solution (Bausch and Lomb); or Opti-Free Enzymatic Cleaner (Allergan, Inc.).

The invention also involves, in another aspect, the promotion of any of the above-mentioned compositions or methods described herein. As used herein, “promoted” includes all methods of doing business including, but not limited to, methods of selling, advertising, assigning, licensing, contracting, instructing, educating, researching, importing, exporting, negotiating, financing, loaning, trading, vending, reselling, distributing, replacing, or the like that can be associated with the methods and compositions of the invention, e.g., as discussed herein. Promoting may also include, in some cases, seeking approval from a government agency to sell a composition of the invention. Methods of promotion can be performed by any party including, but not limited to, businesses (public or private), partnerships, contractual or sub-contractual agencies, educational institutions such as colleges and universities, research institutions, hospitals or other clinical institutions, governmental agencies, etc. Promotional activities may include instructions or communications of any form (e.g., written, oral, and/or electronic communications, such as, but not limited to, e-mail, telephonic, facsimile, Internet, Web-based, etc.) that are clearly associated with the invention. As used herein, “instructions” can define a component of instructional utility (e.g., directions, guides, warnings, labels, notes, FAQs (“frequently asked questions”), etc., and typically involve written instructions on or associated with the composition and/or with the packaging of the composition. Instructions can also include instructional communications in any form (e.g., oral, electronic, digital, optical, visual, etc.), provided in any manner such that a user will clearly recognize that the instructions are to be associated with the composition, e.g., as discussed herein.

Yet another aspect of the present invention provides any of the above-mentioned compositions packaged in kits, optionally including instructions for use of the composition. That is, the kit can include a description of use of the composition for participation in any reaction disclosed herein, for instance, reaction of a lysine or other charged residue of an enzyme or protein with an agent such as an acetylating agent.

A “kit,” as used herein, defines a package including any one or a combination of the compositions of the invention, and/or homologs, analogs, derivatives, enantiomers and functionally equivalent compositions thereof, and the instructions, but can also include the composition of the invention and instructions of any form that are provided in connection with the composition in a manner such that one of ordinary skill in the art would clearly recognize that the instructions are to be associated with the specific composition, for example, as described above. The kits described herein may also contain, in some cases, one or more containers, which can contain compositions such as those described above. The kits may also contain instructions for mixing, diluting, and/or reacting the compositions of the kit. The kits also can include other containers with one or more solvents, surfactants, preservatives, and/or diluents, as well as containers for mixing, diluting, and/or reacting the compositions of the kit.

The compositions of the kit may be provided as any suitable form, for example, as liquid solutions or as dried powders. When the composition provided is a dry powder, the composition may be reconstituted by the addition of a suitable solvent, which is also provided in some cases. In embodiments where liquid forms of the composition are used, the liquid form can be concentrated or ready to use. The solvent will depend on the formulation of the composition and the mode of use.

The following examples are intended to illustrate certain embodiments of the present invention, but do not exemplify the full scope of the invention.

### EXAMPLE 1

Although polyacrylamide gel electrophoresis in the presence of sodium dodecyl sulfate (SDS-PAGE) is a technique that is ubiquitous in biochemistry, the details of the interaction of SDS with proteins are poorly understood. Under the conditions normally used for SDS-PAGE (concentration of SDS=0.1%, ˜3.5 mM), globular proteins associate, on average, with one molecule of SDS per two amino acid residues (or 1.4 g of SDS per 1 g of protein), and upon binding, denature and lose their three-dimensional structure. This broad (although not universal) invariance of the stoichiometry of SDS to protein in the protein-SDS aggregates has the useful consequence that the rate of electrophoretic migration through a matrix of polyacrylamide gel depends on molecular weight.

Denaturation of proteins by SDS is believed to involve both electrostatic and hydrophobic interactions. Most nonionic surfactants do not denature proteins, while ionic surfactants (both positively and negatively charged) with structurally similar hydrophobic tails do; thus electrostatic interactions may play an important role in denaturation of proteins by surfactants.

In this example, the role of electrostatics were investigated in the interaction of a protein and SDS using native carbonic anhydrase (BCA) and its charge-modified derivative (BCA-Ac₁₈) having all of its lysine groups acetylated (ε-NH₃⁺→ε-NH—CO—CH₃). “Ac₁₈” indicates that 18 lysine residues were acetylated. Carbonic anhydrase is a 30 kDa, single-chain protein that is often used as a model in protein biophysics. It does not contain any disulfide bonds. It possesses a mix of alpha-helical and beta-sheet structural elements, with 10 beta-strands forming the core of the protein. Carbonic anhydrase contains a Zn(II) cofactor in the active site; this cofactor (as Zn(II)-OH) may be necessary for its catalytic activity and for binding arylsulfonamide inhibitors. Many inhibitors of carbonic anhydrase are commercially available.

This example investigates the effect of large perturbations in charge, and therefore in electrostatic interactions, on the surface of the protein on its ability to fold into an active form. The folded BCA and BCA-Ac₁₈ were characterized by circular dichroism, activity as an esterase, and binding of an inhibitor. It was shown that a large change in charge on the surface did not perturb the three-dimensional structure of BCA-Ac₁₈. It was found that both BCA and BCA-Ac₁₈ refolded with similar kinetics into the native conformation when the solutions of denatured protein were rapidly diluted to 0.1 mM SDS. Both proteins also refolded when SDS was slowly removed by dialysis. Thus, elimination of a large number of positively charged surface groups by acetylation of lysines did not appear to influence the ability of the protein to refold.

It was observed that BCA-Ac₁₈ denatured more slowly in high concentrations of SDS than BCA. The observed difference between the two proteins may be due to the difference in the thermodynamics of native and denatured forms of the two proteins, or due to the kinetics of denaturation. This example describes detailed examination of the rates of unfolding and refolding of both proteins over a range of concentrations of SDS. It was concluded in this example that BCA-Ac₁₈ is kinetically more stable to denaturation by SDS than BCA.

Sources of chemicals are as follows. Bovine carbonic anhydrase II was purchased from Sigma-Aldrich (St. Louis, Mo.) and used without further purification. SDS was purchased from J. T. Baker (Phillipsburg, N.J.), recrystallized in hot ethanol three times, and stored at −20° C. Dansyl amide, 10× Tris-Gly concentrate, zinc sulfate standard solution, HEPES, and all additives were purchased from Sigma-Aldrich. Dansyl amide (DNSA) was recrystallized once from hot ethanol. The tris-Gly concentrate was diluted 10-fold with deionized water to make 25 mM Tris-192 mM Gly, zinc sulfate (100 micromolar) was added, and the buffer was filtered before use. Dialysis cassettes (10 kDA cutoff) and desalting columns were purchased from Pierce (Rockford, Ill.). Concentrations of BCA and BCA-Ac₁₈ were determined by UV spectroscopy, using ε=57 000 M⁻¹ cm⁻¹ at 280 nm. DNSA was also quantified spectroscopically, using ε=4640 M⁻¹ cm⁻¹ at 325 nm.

BCA-Ac₁₈ was prepared as follows. Peracetylated BCA was prepared without denaturation of the starting protein using published techniques. Briefly, BCA (10 micromolar) was dissolved in 100 mM HEPES buffer. Acetic anhydride was added neat to the solution of BCA in aliquots of 100 equivalents of anhydride with respect to the number of ε-NH₃⁺ groups. The reaction mixture was stirred, and the pH was actively controlled with 1 M NaOH. The extent of the reaction was monitored by capillary electrophoresis (CE) (Beckman P/ACE 5010, 40/47 cm fused silica capillary, 15 kV, Tris-Gly as running buffer) by drawing small aliquots for analysis and desalting them prior to CE. The pH of the reaction mixture was then adjusted to 10.7, the mixture was incubated for 1 h at the elevated pH to deesterify any acetylated tyrosine residues, the pH readjusted to 8.4, and the reaction mixture was dialyzed against Tris-Gly.

Monitoring unfolding of BCA and BCA-Ac₁₈ was performed as follows. The proteins (200 nM) were incubated in different concentrations of SDS in Tris-Gly buffer. A 100 microliter aliquot of protein-SDS solution was mixed with 100 microliters of DNSA solution (30 micromolar) with the same concentration of SDS, and measured the intensity of fluorescence (Perkin-Elmer LS50B spectrometer, λₑₓ=280 nm, λₑₘ=460 nm) as a function of time. The denaturation of BCA was continuously monitored in concentrations of SDS above 4 mM. The rate constants were determined for denaturation by fitting the intensity vs. time data to FU=FUₘₐₓe^{-kt}+FUₘᵢₙ. Unfolding experiments at 37° C. were performed similarly, with samples stored in a dry bath incubator (Fisher Scientific).

Monitoring refolding of BCA and BCA-Ac₁₈ was performed as follows. The proteins (10 micomolar) were denatured in 10 mM SDS for 24 h. CE was used to show that all protein was denatured. Refolding was initiated by dilution of an aliquot of the denatured sample to a final concentration of protein of 200 nM, with varying concentrations of SDS in the buffer. Immediately prior to the measurement, an aliquot of renaturing protein was mixed with an equal aliquot of DNSA. The rate constants for renaturation were determined by fitting the data to FU=FUₘₐₓ(1-e^{-kt})+FUₘᵢₙ. The refolding experiments at 37° C. were performed similarly.

Additives in de-/renaturation included the following. The effects of the following additives on denaturation and renaturation of BCA and BCA-Ac₁₈ were screened: poly(ethelyneglycol) (PEG, MW=2000), 0.1 mM, 1 mM; poly(vinylpyrrolidone), MW=10 000, 1%, 10% w/v; 3-(3-cholamidopropyl)dimethylammoniopropane sulfonate (CHAPS), 1 mM; octanol, 1 mM; tetrabutylammonium thiocyanate, 100 mM; sodium thiocyanate, 100 mM, 1 M; sodium sulfate, 10 mM; betaine, 1 M; dimethyl sulfoxide (DMSO), 1%, 10% v/v; N-methylpyrrolidone, 1%, 10% v/v. The screening was performed in a 96-well plate format, using a SpectraMax Gemini XS spectrometer (Molecular Devices), with a protein concentration of 100 nM, a DNSA concentration of 10 micromolar (present in the denaturation/renaturation buffers), and variable concentrations of SDS.

Dynamic light scattering (DLS) measurements were performed on a DynaPro light scattering device (Wyatt Technology Corp.), at protein concentrations of 10⁻¹⁵ micromolar.

Critical micelle concentration of SDS in Tris-Gly buffer was determined as follows. The critical micelle concentration (CMC) of SDS in 25 mM Tris-192 mM Gly, pH 8.4 buffer was 4.3 mM, as measured by isothermal titration calorimetry. In this example, the kinetics of denaturation and renaturation of proteins in concentrations of SDS below the CMC were studied. The difference in kinetics of denaturation of the two proteins may also occur in concentrations of SDS above the CMC.

BCA-Ac₁₈ was synthesized by reacting the lysine residues of BCA with acetic anhydride in 100 mM HEPES buffer, pH 8.2. The N-terminus of BCA was acetylated posttranslationally. The final product contained ˜90% of BCA-Ac₁₈ and 10% of BCA-Ac₁₇, as determined by peak areas in an electropherogram from separation by capillary electrophoresis. The net charge of BCA in Tris-Gly buffer was about −3, and the net charge of BCA-Ac₁₈ was about −19. It is believed that the difference in charge between BCA and BCA-Ac₁₈ was less than 18 full units of charge due to charge regulation-adjustment of protonation states of other ionizable groups on the protein to counteract the increasing electrostatic potential on its surface.

Binding of DNSA to BCA and BCA-Ac₁₈ was determined as follows. Dansyl amide (DNSA) is an inhibitor of BCA and BCA-Ac₁₈ that is strongly fluorescent when bound in the active site of the protein. In free solution, its quantum yield for fluorescence is 0.055 at λₑₘ=580 nm; when bound to the active site of BCA, its quantum yield increases to 0.84 and the wavelength of maximum emission shifts to 468 nm. The fluorescence of DNSA was used as a measure of the amount of folded protein in solution in the denaturation/renaturation experiments. The presence of SDS (below or above the CMC) did not appear to alter the fluorescence of DNSA in solution. However, it is possible that by using fluorescence of DNSA as a marker for the folded state of the protein, small perturbations in the three-dimensional structure cannot always be observed, if these perturbations do not affect binding of DNSA.

The kinetics of unfolding were determined as follows. The rates of denaturation of BCA and BCA-Ac₁₈ were measured by incubating the protein (200 nM) in solutions of different concentrations of SDS. Immediately prior to the measurement, an aliquot of solution containing protein and SDS was mixed with a solution containing DNSA and the same concentration of SDS; this protocol minimized the possibility that DNSA might stabilize the native conformation of the protein, and influence the rate of its denaturation. When BCA was denatured in concentrations of SDS of 4 mM and higher, DNSA was added to the original, denaturing solution because the rate of denaturation was too fast to follow with manual mixing of aliquots of protein and DNSA. It was observed that DNSA, when present with the protein in the denaturing solution, reduced the rates of denaturation by factors of about 2-10.

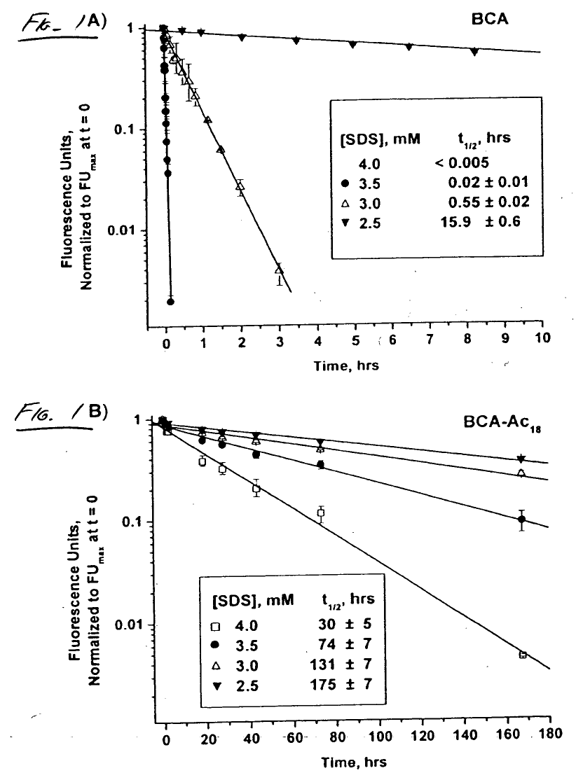

FIG. 1 plots the fluorescence of DNSA, normalized to the signal at t=0, as a function of time of incubation of samples of BCA and BCA-Ac₁₈ in various concentrations of SDS. The decrease in the intensity of fluorescence of DNSA, and thus in the amount of folded protein, followed first-order kinetics. Denaturation of proteins with surfactants probably proceeds through intermediates, but when using a marker that is specific to the folded active site of carbonic anhydrase, transitions beyond the initial unfolding of the three-dimensional structure of the protein were not detected.

BCA appeared to denature in concentrations of SDS at and above 2.5 mM, a value that is substantially below the CMC (4.3 mM in Tris-Gly buffer). In concentrations below 2 mM, it was observed that no denaturation of the proteins over long (˜1 month) periods of time. In high concentrations of SDS, BCA denatured very quickly: t_{1/2} at 4 mM was 15 s (with DNSA present during denaturation), and t_{1/2} at 10 mM was <5 s (faster than the deadtime of the conventional fluorimeter with manual mixing).

BCA-Ac₁₈ denatured in concentrations of SDS at and above 2.5 mM, but with time constants greater than those of BCA by several orders of magnitude. In lower concentrations of SDS (below 2 mM), no denaturation was observed over long (˜1 month) periods of time. In high concentrations of SDS, BCA-Ac₁₈ denatured significantly more slowly than BCA: t_{1/2} at 4 mM was 30 h, t_{1/2} at 10 mM was 160 min; BCA denatured on the time scale of seconds at those concentrations. This difference, approximately a factor of 10⁴, corresponded to a difference of 5 kcal/mol in the activation energy for denaturation of these two proteins. The large differences between BCA and BCA-Ac₁₈ in the rates of denaturation occurred both below and above CMC of SDS, and the presence of the micelles in solution was not necessary to induce unfolding.

FIG. 1 shows unfolding of BCA (FIG. 1A) and BCA-Ac₁₈ (FIG. 1B) as a function of time at various concentrations of SDS, using DNSA fluorescence to measure the concentration of correctly folded protein. The tables list the half-lives (t_{1/2}) for denaturation of BCA and BCA-Ac₁₈ at several concentrations of SDS. The denaturation of BCA at 4 mM occurred very rapidly, and data are not shown to avoid overlap with data at 3.5 mM. The error bars represent the difference between the average and the largest and smallest values measured from at least three replicate measurements.

At concentrations of SDS at and above 2.5 mM, there is a large difference (10¹-10⁴) in the kinetics of denaturation of BCA and BCA-Ac₁₈.

The kinetics of refolding were determined as follows. The rates of refolding of BCA and BCA-Ac₁₈ from the fully denatured states (in 10 mM SDS) were measured by dilution of the solutions into buffers containing concentrations of SDS from 0.1 to 2 mM, using the fluorescence of DNSA as a marker for the appearance of the folded state. At these concentrations of SDS, the proteins exist in folded form (i.e., they do not appear to denature when SDS at these concentrations is added to the folded proteins). As in denaturation studies, an aliquot of solution of the protein and SDS was mixed with an equal aliquot of solution of DNSA with the same amount of SDS immediately prior to the measurement to eliminate the possibility of DNSA influencing refolding kinetics. The buffer also contained 100 micromolar ZnSO₄ to ensure that the folded protein could incorporate a Zn(II) cofactor.

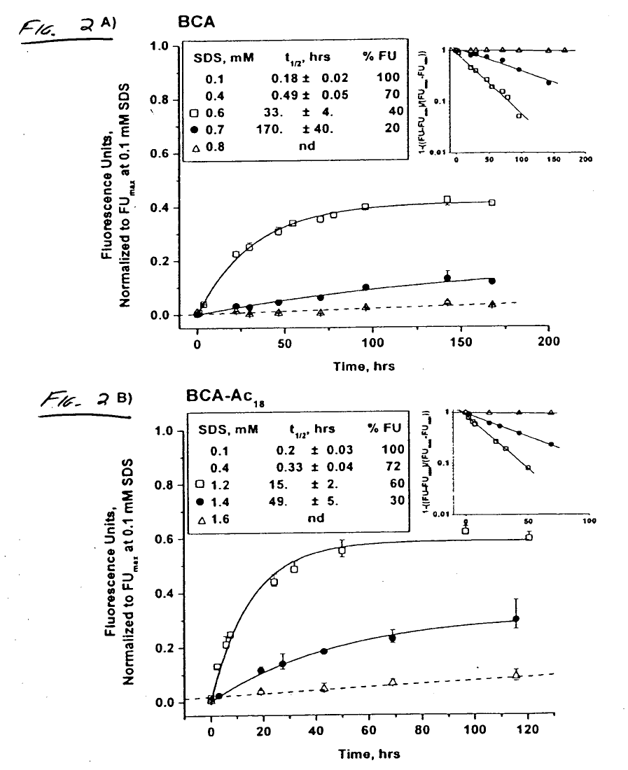

FIG. 2 plots DNSA fluorescence as a function of time after dilution of denatured (10 mM SDS) protein into several concentrations of SDS. The insets show that the increase in fluorescence followed first-order kinetics. Time constants for refolding of both proteins increased with increasing concentration of SDS, but refolding of BCA-Ac₁₈ proceeded faster than refolding of BCA in solutions with the same concentration of SDS. The yields of correctly folded proteins, based on the intensity of the fluorescence signal, decreased with increasing concentration of SDS in the refolding buffer. No refolded BCA at concentrations of SDS of 0.8 mM and higher was observed, or any refolded BCA-Ac₁₈ at concentrations of SDS of 1.6 mM and higher, either due to particularly slow kinetics of refolding or to yields that were too low to be detectable.

FIG. 2 shows refolding of BCA (FIG. 2A) and BCA-Ac₁₈ (FIG. 2B) as a function of time, initiated by rapid dilution of the denatured protein in 10 mM SDS into various concentrations of SDS; the fluorescence signal from DNSA was normalized to that of refolded protein at 0.1 mM SDS. The raw data emphasize the decrease in the yield of folded protein with increasing concentration of SDS. The insets demonstrate that the data fit first-order kinetics. The half-lives (t_{1/2}) for renaturation were determined, where possible, by fitting the data to FU=FUₘₐₓ(1-exp(−t/t_{1/2}))+FUₘᵢₙ; the fits are shown (solid lines). The tables provide data for refolding in 0.1 and 0.4 mM SDS as well as for concentrations on the graphs for comparison. Where the renaturation does not occur and the data cannot be fit to the function above, the symbols are connected by dashed lines. The error bars represent the difference between the average and the largest and smallest values measured from at least three replicate measurements.

The rate-limiting step in the folding of BCA from solutions of GuHCl (guanidinium chloride) occurred with a time constant on the order of 10 min, and may be attributable to proline isomerization. Refolding of BCA from the SDS-denatured state into a minimal (0.1 mM to 0.2 mM) concentration of SDS occurred on a similar time scale, and thus may also be proline isomerization. Because the rates of refolding decreased with increasing SDS in the buffer, it is possible that the rate-limiting step in folding switches from proline isomerization to dissociation of SDS from the polypeptide chainsa process dependent on the concentration of free SDS in the solution.

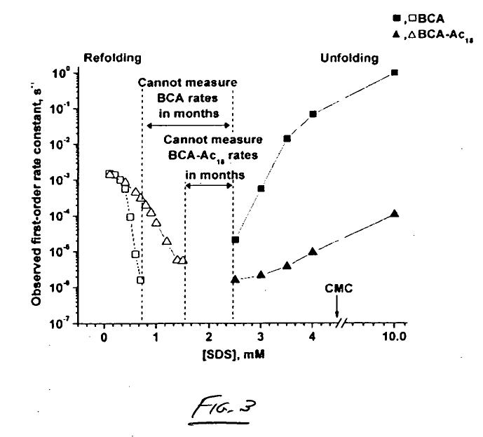

FIG. 3 summarizes the rate constants of unfolding and refolding of both proteins as functions of SDS concentration. This figure shows rate constants for unfolding and refolding of BCA (squares) and BCA-Ac₁₈ (triangles). Closed symbols refer to rate constants for unfolding; open symbols refer to rate constants for refolding. Dashed vertical lines mark the windows in the concentration of SDS where the rates of both unfolding and refolding were not measured. For both proteins, there exists a “window” in which the rates of either unfolding or refolding could not be measured. It is possible that the change in the slope in the rates of refolding, which occurred at 0.3 mM SDS, indicates a change in the rate-limiting step.

Is the native state of BCA or BCA-Ac₁₈ the thermodynamically favored one at intermediate concentrations of SDS? One way to show that a protein is in thermodynamic equilibrium between the native and denatured states at a condition of interest is to approach this condition from both native and fully denatured states. The examination of refolding of BCA and BCA-Ac₁₈ into their native conformations after complete denaturation showed that BCA did not refold at an observable rate in 0.8-2 mM SDS, a range of concentrations in which it also did not unfold at an observable rate. BCA-Ac₁₈ did not refold in 1.6-2 mM SDS, a range of concentrations in which it also did not unfold. In these “windows,” the equilibration between the native and denatured states could not be reached in any practical experiment (time scales of a month). Equilibrium between native and denatured states was not reversible at those intermediate concentrations of SDS, and thus, it could not be concluded that the native conformation of the proteins is the thermodynamically favored state at concentrations of SDS below 2 mM.

Unfolding and refolding experiments were also conducted at 37° C. in an attempt to speed up both rates, and to reach equilibrium. Elevated temperature only shifted the “window” in which equilibration could not be observed, to lower concentrations of SDS; this window of no equilibration could not be eliminated entirely.

The observation that the rate of unfolding of BCA-Ac₁₈ is slower than that of BCA, while the rate of refolding is faster, suggests that the thermodynamic stability of BCA-Ac₁₈ in SDS is greater than that of BCA (i.e., the equilibrium constant for denaturation K_{N-D} of BCA is less than K_{N-D} of BCA-Ac₁₈, based on K_{N-D}=k_{unf}/k_{ref}).

Why is equilibrium not achieved between native and denatured states at intermediate concentrations of SDS? The lack of equilibration between native and denatured states of the protein suggests that some process other than unfolding and refolding becomes competitive at intermediate concentrations of SDS. Aggregation is a process that often interferes with folding of proteins because it occurs between partially folded intermediates. Aggregation is a common feature of beta-sheet proteins, and carbonic anhydrase, in particular, is a protein that is prone to aggregation. It aggregates during renaturation when denatured with heat or acid, or when incubated in intermediate concentrations (1-3 M) of guanidinium chloride (GuHCl).

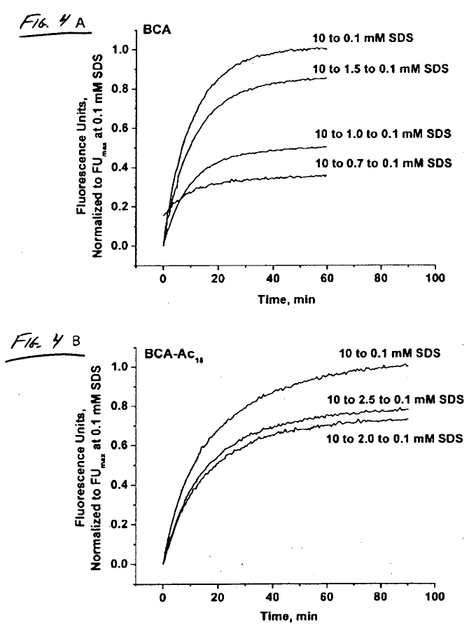

Aggregation was tested by renaturing the proteins from a denatured state at 10 mM SDS to 0.1 mM SDS, but through an incubation step at intermediate concentrations of SDS, instead of renaturing by rapid dilution from 10 mM SDS to 0.1 mM SDS directly (i.e., 10 mM SDS→2.5 mM SDS→0.1 mM SDS instead of 10 mM SDS→0.1 mM SDS). For incubation, concentrations of SDS were chosen in which aggregation was believed to occur on a similar or shorter time scale as refolding. The protein-SDS complex was diluted from 10 mM SDS to 2.5-0.7 mM SDS, the proteins were incubated for 1 week at those concentrations, and the samples further diluted to 0.1 mM SDS to initiate fast refolding. FIG. 4 shows that only a fraction of total protein, as judged by the fluorescence intensity of DNSA, was recovered upon dilution of the incubated samples to 0.1 mM SDS. The reduced yield of the active protein after incubation at intermediate concentrations of SDS suggests irreversible aggregation, where the aggregating species are protein molecules that are not saturated with SDS.

FIG. 4 shows refolding of BCA (FIG. 4A) and BCA-Ac₁₈ (FIG. 4B) after denaturation in 10 mM SDS, incubation of the denatured proteins at intermediate concentrations of SDS (marked for each curve) for 1 week, and dilution into 0.1 mM SDS. The fluorescence signal from DNSA was normalized to that of refolded protein by rapid dilution from 10 to 0.1 mM SDS. All traces showed similar half-lives for refolding (t_{1/2}(BCA)=10.8±0.6 min; t_{1/2}(BCA-Ac₁₈)=17.5±1.4 min), but the yield of folded protein varied with the concentration of SDS in which the samples were incubated.

Dynamic light scattering was also used to measure the sizes of the folded and unfolded protein and of the presumably aggregated sample. The hydrodynamic radius of the native BCA (2.7±0.7 nm), and of BCA, refolded in 0.1 mM SDS (2.6±0.6 nm), were measured. BCA was denatured in 10 mM SDS, and diluted to 1.8 mM SDS, a concentration at which no refoldings were observed immediately before the measurement. The denatured protein resulted in a hydrodynamic radius of 2.9±0.6 nm. Denatured BCA, incubated in 1.8 mM SDS for one week, resulted in a radius of 3.6±0.9 nm. The small increase in the hydrodynamic radius of the incubated sample is indicative of the formation of low-order aggregates (e.g., dimers or trimers), but the resolution of DLS measurements was not sufficient to observe each species individually. These measurements suggested that multimers formed on refolding of BCA in intermediate concentrations of SDS are similar in size to those forming in GuHCl.

The yields of refolded BCA-Ac₁₈ after incubation were higher than the yields of BCA. Since refolding of BCA-Ac₁₈ proceeded to higher concentrations of SDS than refolding of BCA, these observations may indicate differential propensities for aggregation between the two proteins. BCA-Ac₁₈ may aggregate less due to electrostatic repulsion of highly negatively charged molecules.

In some experiments, various additives were used to prevent aggregation of BCA and BCA-Ac₁₈. These additives were commonly used to prevent aggregation of carbonic anhydrases and other proteins. The additives included polymers (PEG), other surfactants (CHAPS), “salting-in” Hofmeister salts (tetrabutylammonium thiocyanate and sodium thiocyanate), osmolytes (betaine), and organic solvents (dimethyl sulfoxide and N-methylpyrrolidone). These additives shifted the “window” in which neither unfolding nor refolding was observed, but did not help achieve equilibration at all concentrations of SDS; the shift in the “window” appeared to be linked to the shift of critical micellar concentration of SDS due to the additives. For example, in 10% DMSO, it was observed that refolding of BCA up to 1.2 mM SDS (vs. 0.7 mM SDS without a cosolvent), but unfolding up to 2.5 mM SDS was not observed. In the presence of 1 mM CHAPS, the refolding of BCA occurred up to 1.8 mM SDS, but unfolding did not occur below 2.5 mM SDS.

In conclusion, either the net charge of BCA or the number of positively charged residues influences the kinetics of its denaturation with SDS. The example of BCA (net charge of about −3, 18 lysine —NH₃⁺ groups) and peracetylated BCA (net charge of about −19, all lysines converted to —NH—CO—CH₃ groups) showed that the rates of denaturation with SDS of these proteins can change by −3 orders of magnitude as a result of a change in the charge of the protein by a factor of 5.

This example shows that structural features of proteins are not the only determinants of kinetic stability of proteins to SDS. BCA and BCA-Ac₁₈ have similar core structures based on CD spectra, yet the large negative charge of BCA-Ac₁₈ relative to BCA renders the protein kinetically more stable than BCA to negatively charged SDS by reducing the rate of denaturation.

Thus, the experiments in this example demonstrate that equilibration in protein/surfactant systems may occur on long times scales (days to months); this phenomenon can occur both below and above the CMC of the surfactant. Proteins and their derivatives may behave differently toward SDS, especially if the derivative differs from the native protein in charge.

### EXAMPLE 2

One goal of this example was to distinguish between the effects of charge and of hydrophobicity on the kinetics of denaturation of a model protein, bovine carbonic anhydrase (BCA, E.C. 4.2.1.1), and of derivatives of this protein generated by acylation, having different charges and hydrophobicities.

BCA is a good model for studying processes involving denaturation. BCA is easy to handle, monomeric, and commercially available; it has no disulfide bonds. Its structure is well-defined by X-ray crystallography, and its binding of arylsulfonamide inhibitors is also structurally well-defined. A wide range of those inhibitors is commercially available and synthetically accessible. There is literature describing the denaturation of BCA with other denaturants, i.e., urea and guanidinium chloride (GuHCl). These studies generally show that the rate of folding of BCA can be determined by the isomerization of proline residues, that BCA is not completely unfolded, even in saturated solutions of GuHCl, and that BCA, like many proteins that have a large fraction of its structure in beta-sheets, may be susceptible to aggregation in the partially (un)folded state.

The denaturation and renaturation of BCA with SDS is reasonably well-characterized: it is reversible at low concentrations of SDS (<0.1 mM), and can be followed by capillary electrophoresis (CE). Example 1 illustrates synthesis of a derivative of BCA, BCA-Ac₁₈ (all 18 lysine groups acetylated), in which the tertiary structure of the proteins is indistinguishable (by catalysis and circular dichroism) from the native structure, but where the external surface of the proteins lacks all 18 of the positively charged lysine ε-NH₃⁺ groups present in the native protein. After denaturation with SDS, both BCA and BCA-Ac₁₈ refold with similar rates (11±1 min for BCA and 21±2 min for BCA-Ac₁₈) to the same (native) structure upon complete removal of the SDS, as discussed above.

Zn(II) cofactor of carbonic anhydrase does not complicate studies of refolding. The Zn(II) cofactor is not required for refolding into a native-like conformation, does not remain associated with the unfolded protein, and does not significantly change the rate of refolding. The presence of the Zn(II) cofactor during refolding, however, does increase the total amount of recovered protein by a factor of 2. All of the solutions used in this example contain 100-micromolar Zn(II), so that any folded BCA or BCA-derivative contains the Zn(II) cofactor (and therefore binds inhibitors).

Protein charge ladders allows systematic variation of protein charge. Reaction of BCA with limited quantities of an anhydride converted some of the 18 lysine-ε-NH₃⁺ groups to lysine-ε-NHCOR groups. The derivatives appeared in CE as a set of peaks with regular spacing, i.e., a protein charge ladder. Each “rung” of the charge ladder (with the exception of the native and completely acylated forms) is believed to be a set of regioisomers with the same number, but different distribution, of modified lysine residues, and therefore, approximately the same net charge. Charge ladders are thus a family of derivatives of a protein, in which the charge can be changed systematically. Using different acylating reagents, another parameter can be independently varied—hydrophobicity—and charge can be used to count the number of modifications. Variation in the extent of acylation, and in the structure of the acylating reagent, allow charge and hydrophobicity to be changed independently.

A comparison of rates of denaturation of acetyl- and hexanoyl-charge ladders of BCA is as follows. In this example, the rates of denaturation of two charge ladders of BCA were compared, one prepared with acetic anhydride, (CH₃CO)₂O (“BCA-Acₙ”), and one prepared with hexanoic anhydride, (CH₃(CH₂)₄CO)₂O (“BCA-Hexₙ”). The kinetics of denaturation of both were studied since the rates of denaturation and renaturation were intractably slow at the intermediate (1-2.5 mM) concentrations of SDS that would be required for equilibration. In addition, another process, presumably aggregation of partially folded intermediates, may occur at low concentrations of SDS (0.7-2.5 mM) and prevents equilibration of the folded and denatured states.

All members of the charge ladders used in this example were stable at room temperature in the absence of denaturant. Each rung of both of the charge ladders used here binds sulfonamide inhibitors (K_{d}˜0.3-1.3 mM). It was concluded, as discussed below, that all of the rungs retained a common active-site structure, and a common tertiary structure.

For any charge ladder of BCA, as the number of modifications (n) increases, the total charge on the protein becomes more negative, and the surface of the protein becomes more hydrophobic due to conversion of NH₃⁺ groups to NHCOR groups. Native BCA has a charge of ˜−2.9 at pH 8. The charge on the early rungs of the ladder increased linearly with the number of acylations; each rung adds an additional charge of ˜−0.9. Therefore, for example, BCA-Ac₈ has a charge of ˜−10. The later rungs of the ladder may differ by <0.9 units of charge. Using the Linderstrom-Lang model of cooperativity in proton binding, the charge on BCA-Ac₁₈ was calculated to be −19. The mobility of a given rung of BCA-Acₙ may have nearly the same mobility as the corresponding rung of BCA-Hexₙ. Thus, the charges of the BCA-Hexₙ may be indistinguishable from those of BCA-Acₙ for a given rung number. (Small deviations in mobility between later rungs of the two ladders may be due either to a change in charge or a change in drag between the two ladders. It can be assumed that any differences in the mobilities are due to additional drag from the hexanoyl groups relative to the acetyl groups.

Hydrophobicity parameters (Hansch pi-parameters, log P-values) are often used to quantify the hydrophobicity of modifications to molecules. The change in hydrophobicity from NH₃⁺ (log P=−2.12) to NHCOCH₃ (one modification using acetic anhydride, log P=−1.21) was +0.9, that is, more hydrophobic. The change in log P for the change from NH₃⁺ to NH(COCH₂)₄CH₃ (one modification with hexanoic anhydride) was +2.9.

Capillary electrophoresis was used to monitor the denaturation of charge ladders of BCA. As the negatively charged SDS molecules interact with the proteins, the electrophoretic mobility of the complex increased above that of BCA-Ac₁₈; all of the rungs of both of the ladders had indistinguishable mobilities when denatured with SDS. Because the mobilities of the denatured proteins—fully associated with SDS—were much larger than the mobilities of any folded proteins, all of the rungs of the charge ladder and the denatured protein in the same sample could be observed. Proteins were detected using absorption at 214 nm; at this wavelength, the amide bonds and aromatic side chains of the protein were expected absorb, but the SDS is transparent. Thus, there would be no interference from micelles of SDS, and CE can be used with SDS both above and below the critical micelle concentration (CMC).

A model of the kinetics of the interaction of charge ladders of BCA with SDS follows. The kinetics of denaturation were studied using transition state theory (Eq. 1):

k=νκ_{TST}e^{-ΔG}^{/RT} (1).

In this equation, the experimentally measured rate (k) could be related to the activation energy (ΔG^{t}), or the difference in energy between the folded, starting state, and the conformations in the saddle point of the reaction. In Eq. 1, ν is a characteristic vibration frequency along the reaction coordinate at the saddle point and κ_{TST} is the transmission coefficient. For simple chemical reactions, κ_{TST} is often assumed to be 1; that is, all of the molecules passing through the transition state proceed to product, and ν=k_{b}T/h (˜6×10¹² s⁻¹) where k_{B} is the Boltzmann constant, T is the temperature, and h is the Plank constant. For protein folding, however, the transmission through the saddle point is believed to be much less than unity. An empirical estimate for νκ_{TST} in the folding of proteins is 10⁶ s⁻¹. This number was calculated for cytochrome c (t_{folding}˜400 ms) and is, probably, an underestimate for BCA because it is a larger protein than cytochrome c and folds much more slowly (t_{folding}˜10 min). Assuming that νκ_{TST} does not change with type or number of acylation, an incorrect estimate for νκ_{TST} will affect only the scale of ΔG^{‡}. An underestimate in the value of νκ_{TST} will lead to an overestimate in the value of ΔG^{‡}.

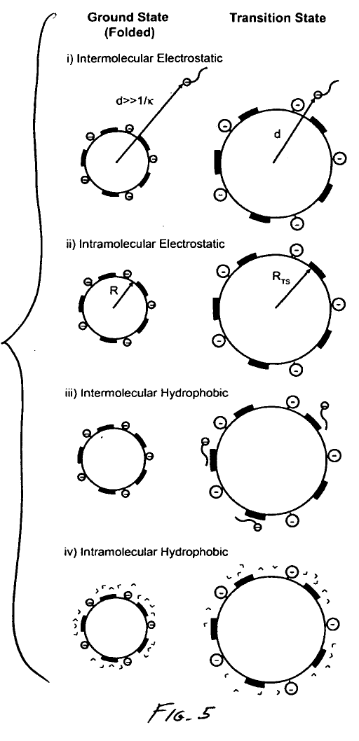

Following is a qualitative description of the model of SDS-protein interaction. It is proposed that each acylation influences the activation energy, and thus the rate of denaturation of the rungs of charge ladders of BCA in four ways (see schematic diagram in FIG. 5). FIG. 5 is a schematic representation of the four factors (discussed below) for the model of protein denaturation. The protein is represented as a sphere with uniformly distributed negative charge on its surface. The dark patches represent hydrophobic regions on the surface of the protein that result from acylations. The depictions of SDS molecules are wavy lines (dodecanoic chain) with negatively charged headgroups (sulfate group). V-shaped entities represent water molecules.

i. Intermolecular electrostatic interaction. Each acylation increases the electrostatic repulsion between the more negatively charged proteins and the negatively charged SDS relative to the electrostatic repulsion between BCA and SDS. The increased electrostatic interaction increases the stability (and therefore, ΔG^{‡}) of the latter rungs of the charge ladder, relative to unmodified BCA, to denaturation by SDS.

ii. Intramolecular electrostatic interaction. Each acylation decreases the stability of the folded protein, relative to BCA, by increasing the net charge on its surface. The charge-charge repulsion destabilizes the folded state of a protein relative to BCA and makes the latter rungs of the charge ladder less stable than the early rungs to denaturation by SDS. The intramolecular electrostatic repulsion decreases ΔG^{‡} of each rung of the charge ladder relative to ΔG^{‡} of BCA.

iii. Intermolecular hydrophobic interaction. Each acylation also increases the exposed hydrophobic surface area and destabilizes the folded protein relative to BCA due to an increase in the interaction between the protein and the hydrophobic tails of the SDS molecules.

iv. Intramolecular hydrophobic interaction. Each acylation destabilizes the folded protein relative to BCA due to an increase in exposed hydrophobic surface area and an increased ordering of water in the folded state relative to unmodified BCA. Both of the effects of increased hydrophobic surface area (effects iii and iv) should make the latter rungs of the charge ladder less stable than the early rungs to denaturation with SDS.

This model does not account for any specific (local) interactions that are created or destroyed by acylation (e.g., removal of salt bridges between lysine and other anionic residues on the protein, steric interactions caused by increasing the size of the lysine residue, or specific interactions between positively charged residues and molecules of SDS); it treats the protein as a distribution of charges and hydrophobic surface area. Positively charged residues on the surface of the protein may provide places for the negatively charged SDS molecules to bind and nucleate further unfolding. This is neglected in this example. Although neglecting local interactions runs the risk of neglecting important specific interactions, it is demonstrated that this model replicates the trends in the data without using them. There is thus no need, at least for BCA, to consider local interactions to describe how the rate of denaturation changes with acylation of lysine residues.

The relative importance of these stabilizing (electrostatic) and destabilizing (hydrophobic and electrostatic) effects were quantified as the number of modifications increased. In comparing rungs with the same number of modifications, n, across charge ladders made with different anhydrides, the charge remains the same, but the hydrophobicity differs. It is assumed that the patterns and regioselectivity of acylation with acetyl and hexanoyl ladders are similar. Thus, the effects of charge and hydrophobicity in one experimental system can be distinguished.

In this example, all chemicals were reagent grade unless stated otherwise. Acetic anhydride, hexanoic anhydride, bovine carbonic anhydrase, 10× Tris-Gly concentrate, HEPBS, dioxane, and dimethylformamide were purchased from Sigma Chemical (Milwaukee, Wis.). Dialysis cassettes (weight cutoff of 10 kDa) and desalting spin columns were purchased from Pierce (Rockford, Ill.). SDS (Baker Chemical, Phillipsburg, N.J.) was recrystallized in hot ethanol three times, then dried and stored at −20° C. until use. SDS was discarded or repurified after 2 months. Tris-Gly buffer was made by diluting 100 mL of the 10× concentrate with 900 mL of freshly distilled, deionized water and filtered with a 0.22-micrometer filter (Pall, Ann Arbor, Mich.) before use.

Protein modification using hydrophobic anhydrides was prepared as follows. Solutions were made of 100 micromolar of BCA in 500 microliters of 0.1M HEPBS buffer, pH 9. Stock solutions of anhydrides were made by diluting 10 microliters of anhydride (acetic or hexanoic) into 500 microliters dioxane. This stock solution was then diluted with dioxane to make concentrations of anhydride that were 6, 12, and 18 times the concentration of lysines (1.9 mM) in the reaction mixture. These reagents were made immediately before they were used. Twenty-five microliters of each of the diluted stocks of anhydride were added to the protein solutions, so that the final ratio of anhydride to lysine was 0.3, 0.6, and 0.9 in each of the reaction mixtures. The mixtures were agitated immediately using a vortex mixer. The reactions were left overnight to ensure complete reaction. The proteins were desalted into 1× Tris-Gly buffer using spin desalting columns. Each reaction mixture was then run on CE to determine the relative concentration of each rung. The reaction mixtures were then combined so that the final concentration of each rung was approximately constant across the ladder.

Denaturation experiments were performed as follows. A charge ladder of BCA (BCA-Acₙ or BCA-Hexₙ, 1 mL of 100 micromolar total protein in Tris-Gly buffer) was placed in a dialysis cassette (MW cutoff of 10 kDa). The dialysis cassette was placed in a 1 L bath of 3 mM SDS in Tris-Gly buffer at room temperature. The buffer was changed every 24 h. At regular intervals of time (approximately every half-hour), 100 microliters of the protein solution was removed from the dialysis cassette; ˜7 microliters of that aliquot was diluted 10-fold and the absorbance of the diluted sample at 280 nm (ε_{280,BcA}=57,000 M⁻¹ cm⁻¹, the absorption cross section can be assumed to be is unchanged by acylation) was measured to determine the total protein concentration. The aliquot was diluted because the absorbance of the 100 micromolar solution was too high to be read accurately by the ultraviolet spectrometer. This diluted aliquot was discarded. The remainder of the solution was then run on CE. The protein solution, except for the portion diluted for ultraviolet measurement, was returned to the dialysis cassette; the aliquot was outside of the dialysis cassette for ˜5 min.

Capillary electrophoresis experiments were carried out in a Beckman (Fullerton, Calif.) PACE-MDQ system, using a capillary of inner diameter of 50 micrometers of total length of 110.2 cm, 100 cm to the detector. Tris-Gly in D₂O was used as the running buffer, and the applied voltage was 30 kV. D₂O was used in place of H₂O for the electrophoresis buffer because the viscosity of D₂O is higher than that of H₂O and the higher viscosity minimizes diffusion. Samples were injected using pressure (20 psi) for 30 s. Each sample contained 0.65 mM dimethylformamide as an electrically neutral marker for electroosmotic flow.

The CMC of SDS in the Tris-Gly buffer used in this example was 4.3 mM. 3 mM SDS was chosen for these experiments because this concentration of SDS denatured BCA in an interval of time that was convenient for experimental work using CE. In addition, this concentration of SDS was close to the concentration often used in SDS-PAGE (0.1% or 3.5 mM). 1 mL of a solution of ˜100 micromolar of a charge ladder of BCA, dissolved in Tris-Gly buffer, was placed into a dialysis cassette (molecular weight cutoff of 10 kDa) and the cassette placed in 1 L of Tris-Gly buffer containing 3 mM SDS. The bath was kept at room temperature (˜22° C.) for the duration of the experiment.

SDS molecules passed through the dialysis cassette, but the protein and protein-SDS aggregates and SDS micelles did not. The dialysis cassette was used to maintain a constant concentration of free SDS in the solution around the protein. If it is assumed that BCA and its charge variants, like most proteins, bind SDS at a ratio of ˜1 SDS molecule per 2 amino acids, each BCA molecule should bind ˜130 SDS molecules. Because BCA binds so many molecules of SDS, it is difficult to keep the concentration of free SDS constant without a large source (here using a dialysis cassette). Without a dialysis cassette, denaturation would need to be studied at concentrations of SDS more than 130 times the protein concentration (100 micromolar in these experiments) i.e., above 13 mM. With the dialysis cassette, molecules of SDS could be added to the system without changing the concentration of free SDS, and to study the denaturation of proteins below the CMC of SDS.

At regular intervals of time (approximately every half hour), 100-microliter aliquots of the solutions containing the charge ladder of BCA were removed from the cassette in a bath of 3 mM SDS in Tris-Gly buffer, the total protein concentration in the aliquot was measured using absorbance at 280 nm, and a portion (˜10 nL) of the aliquot injected onto the CE. The unused portions of the aliquots were then returned to the cassette. The aliquots were out of the dialysis cassette for <5 min; this time is shorter than the shortest times for denaturation (˜16 min) measured in these experiments for denaturation, and therefore should not significantly affect these measurements.

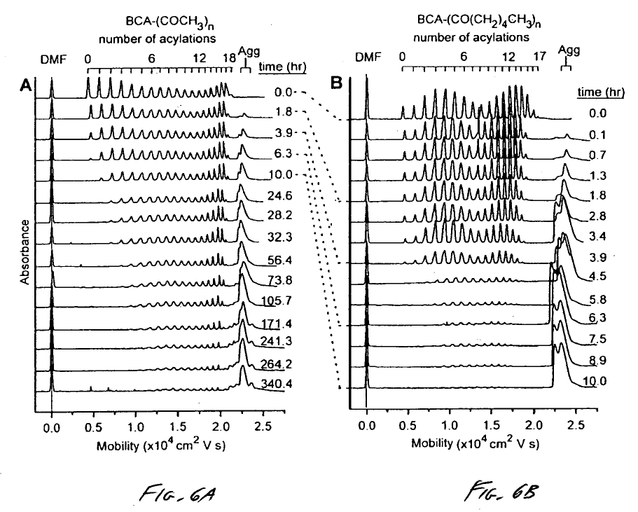

FIG. 6 shows electropherograms as a function of time for the acetyl- and hexanoyl charge ladders of BCA. To quantify the rate of denaturation of each rung of the charge ladders, the peaks were integrated and then corrected for three factors. FIG. 6 shows denaturation of hydrophobic charge ladders of (FIG. 6A) BCA-Acₙ and (FIG. 6B) BCA-Hexₙ. Dimethylformamide was used as a neutral marker of electroosmotic flow. Each ladder is labeled with the time elapsed after placing the dialysis cassette containing protein in the solution of SDS; the dotted lines match up measurements of BCA-Acₙ and BCA-Hexₙ measured at the same amount of elapsed time. The peak corresponding to denatured, aggregated BCA-SDS is labeled (Agg) and has fine structure; this structure may be due to different denatured states of the BCA-SDS aggregate.

Residence time in the detection volume was determined as follows. Proteins that had a higher velocity along the capillary spend less time in the detection volume than proteins that have lower velocity. If two proteins with the same absorptivity were present in a sample in equal concentrations, the protein of lower velocity would have a larger measured peak area than the protein of higher velocity. To correct for this experimental bias, the area of each peak was multiplied-by the velocity of the protein (A_{corr}=A_{measured}×L_{D}/t_{D}), where L_{D} is the length of the capillary from the end (where injection occurs) to the detector (100 cm in all of these experiments) and t_{D} is the time it takes for the rung to reach the detector.

Initial differences in concentrations of each rung were as follows. The areas of each of the rungs in the charge ladder were not all equal before denaturation. To measure the fraction of each rung that has denatured at each time, the velocity-corrected area of each rung in the sample was divided by the velocity-corrected area of that rung in the charge ladder run in the absence of SDS.

The total protein concentration, as measured by absorbance at 280 nm, decreased by ˜15% over a week of dialysis, presumably through association with the dialysis membrane, and/or leakage out of the dialysis cassette. The correction for total protein concentration assumes that regardless of the mechanism by which protein is lost, each rung is lost equally.

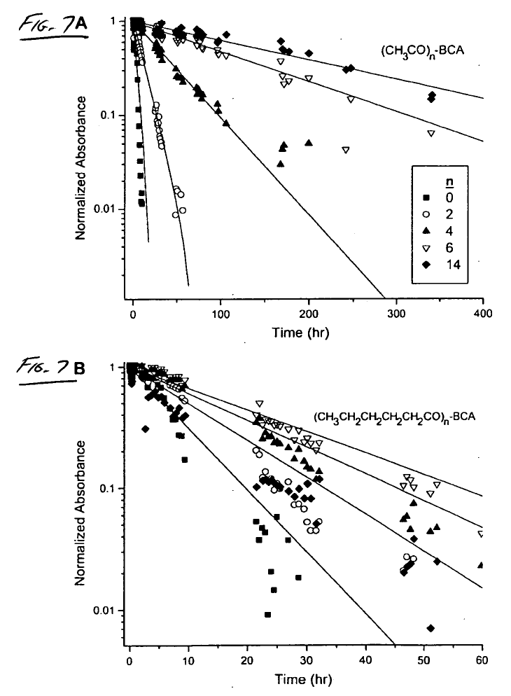

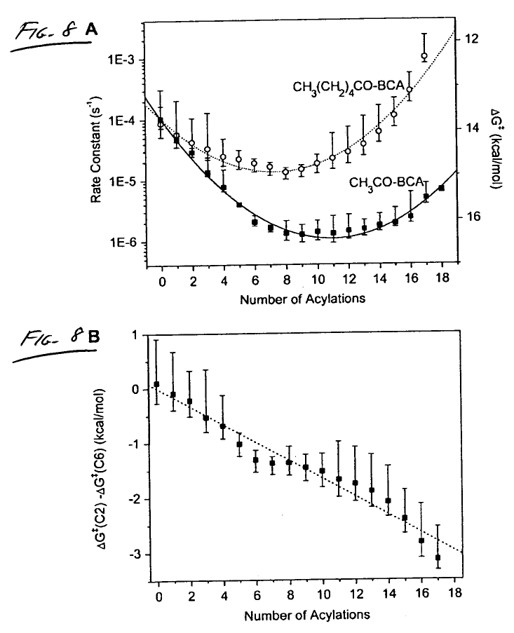

A comparison of the rates of denaturation follows. FIG. 7 shows the decrease in corrected peak area as a function of time for representative rungs of the BCA-Acₙ and BCA-Hexₙ charge ladders. FIG. 7A shows an BCA-Acₙ charge ladder and FIG. 7B shows a BCA-Hexₙ charge ladder. Deviations from linearity could be due to the fact that each rung of the charge ladder is made up of a mixture of regioisomers that may have different rates of denaturation. The data shown in this figure are from one experiment. Each of these sets of data was fit to a single exponential decay and the rate of denaturation as a function of rung number was plotted (FIG. 8A).

FIG. 8A shows rate constants of denaturation for both (squares) BCA-Acₙ and (circles) BCA-Hexₙ charge ladders with SDS as a function of the number of acylations. The points are the arithmetic average and the error bars are minimum and maximum values measured in three repetitions for BCA-Acₙ and four repetitions for BCA-Hexₙ. The right y axis shows the corresponding ΔG^{‡} in kcal/mol calculated using Eq. 1. The lines show fits of the equation ΔG^{‡}=a+bn+cn² to the data. FIG. 8B is a plot of the difference in ΔG^{‡} between a rung of an acetyl ladder and a hexanoyl ladder as a function of rung number. The data fit a line (slope=0.17 kcal/mol, R²=0.97). The fit of these data to a linear plot (dotted lines) suggests that the difference in ΔG^{‡} between the two ladders is only due to the difference in the linear term bn; the c coefficient includes only electrostatic contributions to ΔG^{‡}.

In principle, since every rung (except the native—BCA—and fully functionalized proteins—BCA-Ac₁₈) is a collection of regioisomers, the denaturation profile may not be a single exponential function. The single exponential was used as a measure of the relative rates of denaturation of each rung. The exact functional form is not required for this analysis, and each set of the data in FIG. 3 appears to fit a single exponential well. FIG. 8A shows that there is a pronounced minimum in the plot of the rates of denaturation versus number of acylations for both charge ladders; the earlier and later rungs denature more rapidly, and the middle rungs denature least rapidly.

Following is a mathematical model of the kinetics of the interaction of charge ladders of BCA with SDS. As described above, a model is used in this particular example in which there are two types of interactions—ectrostatic and hydrophobic—that change the ΔG^{‡} for denaturation of modified BCA by SDS relative to that of BCA. Each of the two types of interactions has an intermolecular component that describes how the modification changes the interaction between SDS and protein, and an intramolecular component that describes how the modification changes the stability of the modified BCA in the absence of SDS. This mathematical description of the model fit the data describing rate of denaturation versus the number of acylations (FIG. 8A).

The four types of interactions should add to give the total activation energy of denaturation for each rung of a charge ladder (Eq. 2),

$$Δ {G}_{BCA - Xn}^{‡} = \begin{matrix}Δ {G}_{BCA}^{‡} + ΔΔ {G}_{e - , p - SDS}^{‡} + ΔΔ {G}_{e - , p}^{‡} + \\ ΔΔ {G}_{hydrop - SDS}^{‡} + ΔΔ {G}_{hydrop}^{‡}\end{matrix} , \qquad ( 2 )$$

where ΔG^{‡}_{BCA-Xn} is the activation energy of denaturation for the nth rung of a charge ladder, ΔG^{‡}_{BCA} is the activation energy of unmodified BCA, and the other terms are the additional activation energies of unfolding, relative to BCA, due to i), intermolecular electrostatic repulsion between the SDS molecule and the modified BCA (ΔΔG^{‡}_{e-,p-SDS}); ii), intramolecular electrostatic repulsion between the charges on the surface of the modified BCA (ΔΔG^{‡}_{e-,p}); iii), intermolecular hydrophobic interaction between the SDS molecules and modified BCA (ΔΔG^{‡}_{hydro p-SDS}); and iv), intramolecular hydrophobic interaction due to the additional exposed hydrophobic residues on the surface of modified BCA (ΔΔG^{‡}_{hydro,p}). To write down a functional form for each of these interactions, and to build a tractable model, a number of approximations can be used to simplify the system composed of the protein, surfactant, and aqueous solvent.

The assumptions in the mathematical description of the model were as follows. Both the structure of the folded protein and the structure of the transition state can be approximated as spheres with net charge uniformly distributed on the surface, and with a uniform dielectric constant inside this shell of charge. The model in this example ignores all molecular-level details of protein, surfactant, and the solvent. The number (and distribution) of the molecules of SDS that bind to the protein in the transition state are also considered to be constant for all rungs of the charge ladders. This assumption concerning the stoichiometry of the transition state is suspect, but required to write an equation for the intermolecular electrostatic repulsion term; a single value is assumed for the number of SDS molecules bound in the transition state (m) for all BCA derivatives. The value of dielectric constant of water (ε_{w}) is assumed to be 80, and the change in the dielectric constant that probably occurs near the surface of the protein is neglected. The dielectric constant of water may be close to 20 over a few layers of water molecules (a few angstroms) due to the reduced mobility of the water molecules next to the surface of the protein. The dielectric constant may be affected by the distribution of charged, polar, and apolar groups in the protein and the net charge of the protein. It is further assumed that the dielectric constant is uniform throughout the interior of the protein (εₚ=5). In real systems, the dielectric constant is structured on a microscopic scale and may vary with position. The dielectric constant of solvent-exposed regions of the protein is probably higher than the interior due to configurational mobility of polar side chains.

Each conversion of a lysine-ε-NH₃⁺ group to a lysine-ε-NHCOR group changes the charge (ΔZ) by <1 unit of charge, reflecting charge regulation. The value of ΔZ is close to −0.9 for the first few acylations in the conditions used here (pH 8.4), and is probably <−0.9 (probably between −0.7 and −0.9) at high numbers of acylation. It can be assumed that ΔZ=−0.9 for all acylations regardless of the acylating reagent and number of prior acylations (that is, for example, it can be assumed that BCA-AC₅ and BCA-Hex₅ have the same net charge and charge distribution). In these calculations, only first-order electrostatic interactions are considered. Higher-order electrostatic interactions (for example, charge-dipole and charge-induced dipole effects) between the charged surfactant and protein (assumed to be a dielectric sphere) are ignored.

This model is a major simplification, relative to the real proteins. Proteins are not a spherical shell of charges—not all of the charges are uniformly distributed or located at the surface of the protein, and the protein can compensate for additional charges by changing the values of pKₐ of nearby groups. Using these assumptions, however, equations for each of the terms in Eq. 2 can be derived.

Intermolecular electrostatic repulsion between SDS and BCA. The repulsion between a negatively charged molecule of SDS and a protein can be described by Coulomb's law in water containing salts (Eq. 3):

$$E = \frac{{q}_{SDS} {q}_{BCA - xn}}{4 {πɛ}_{0} {ɛ}_{w} d ( 1 + κ d )} , \qquad ( 3 )$$

where q_{SDS} is the charge on SDS (−1 e_{c}), e_{c} is the charge of an electron, q_{BCA}_{_{—}}_{Xn} is the charge on the nth rung of the charge ladder, ε_{w} is the dielectric constant of water, ε₀ is the permittivity of free space, d is the distance between the center of the sphere representing the protein and the molecule of SDS, and κ is the inverse Debye length (0.333 nm⁻¹ in Tris-Gly buffer, ionic strength of 10 mM). The ΔΔG^{‡}_{e-,p-SDS} term (the difference between ΔG^{‡}_{e-,p-SDS} for BCA-Xₙ and for BCA) is then given by Eq. 4:

$$\begin{matrix}ΔΔ {G}_{e - , p - SDS}^{‡} = ΔΔ {G}_{e - , p - SDS}^{‡} ( BCA - {X}_{n} ) - ΔΔ {G}_{e - , p - SDS}^{‡} ( BCA ) \\ = \frac{{mq}_{SDS} {q}_{BCA - {x}_{n}}}{4 {πɛ}_{0} {ɛ}_{w} d ( 1 + κ d )} - \frac{{mq}_{SDS} {q}_{BCA}}{4 {πɛ}_{0} {ɛ}_{w} d ( 1 + κ d )} \\ = \frac{- {me}_{c}^{2} Δ Zn}{4 {πɛ}_{0} {ɛ}_{w} d ( 1 + κ d )} ,\end{matrix} \qquad ( 4 )$$

where q_{BCA} is the charge on native BCA (Z₀=−3 e_{c}), and m is the number of molecules of SDS that are bound to the protein in the transition state.

Only values of free energy are considered in this calculation. In water, the relative partitioning of the free energy due to Coulombic interactions into enthalpy and entropy is complicated, and the majority of the free energy may be due to entropy (not due to enthalpy—the major portion of the free energy in vacuum). In the interaction of protein with molecules of SDS, it is unclear whether the solvation (entropic effects) or enthalpy is the primary contributor to ΔG.

Intramolecular charge/charge repulsion is as follows. The change in energy upon adding a charge to a uniform shell of charge on the surface of a protein is given by Eq. 5,

$$\begin{matrix}ΔΔ {G}_{e - , p}^{‡} = \frac{{q}_{BCA - Xn}^{2} - {q}_{BCA}^{2}}{8 {πɛ}_{0} {ɛ}_{p} R ( 1 + κ R )} - \frac{{q}_{BCA - Xn}^{2} - {q}_{BCA}^{2}}{8 {πɛ}_{0} {ɛ}_{w} {R}_{TS} ( 1 + κ {R}_{TS} )} \\ = \frac{{{e}_{c}^{2} ( {Z}_{0} - n Δ Z )}^{2} - {e}_{c}^{2} {Z}_{0}^{2}}{8 {πɛ}_{0} {ɛ}_{p} R ( 1 + κ R )} - \frac{{{e}_{c}^{2} ( {Z}_{0} - n Δ Z )}^{2} - {e}_{c}^{2} {Z}_{0}^{2}}{8 {πɛ}_{0} {ɛ}_{p} {R}_{TS} ( 1 + κ {R}_{TS} )} \\ = \frac{{e}_{c}^{2}}{8 {πɛ}_{0} {ɛ}_{p}} ( \begin{matrix}\frac{1}{R ( 1 + κ R )} - \\ \frac{1}{{R}_{TS} ( 1 + κ {R}_{TS} )}\end{matrix} ) \times ( - 2 {Z}_{0} Δ Zn + {n}^{2} ) ,\end{matrix} \qquad ( 5 )$$

where R is the radius of the protein, εₚ is the dielectric constant in the interior of the protein, and R_{TS} is the radius of the transition state. This equation approximates both the folded protein and the transition state of the protein-SDS aggregate as spheres.

Because it has been assumed that ΔZ is the same for acylations in all positions, and because it has been assumed that the charge is uniformly distributed on the surfaces of the spherical protein, proteins contained in a given rung of the charge ladder, even though they are regioisomers, should repel a molecule of SDS with the same force. The two equations (3 and 5) that describe how changes in electrostatics affect changes to ΔG^{‡} will, therefore, be the same for both the acetyl and hexanoyl charge ladders.

Hydrophobic contributions to ΔG^{‡} are as follows. Intermolecular hydrophobic interaction between exposed hydrophobic surface area of BCA and molecules of SDS. The additional exposed hydrophobic surface area on the acylated proteins relative to BCA increases the interaction (and hence the equilibrium constant for association) between SDS molecules and the protein. It can be assumed that this increase in interaction is proportional to the additional hydrophobic surface area of the acylated proteins relative to unacylated BCA (Eq. 6):

ΔΔG^{‡}_{hydro,p-SDS}=C_{hydro,p-SDS} n (6).

In this equation n is the number of modifications, and C_{hydro,p}_{_{—}}_{SDS} is a constant of proportionality that is larger for hexanoyl than for acetyl ladders. The ratio of the pi-parameters described in the introduction suggests that each modification with hexanoic anhydride results in a change in hydrophobicity that is similar to three acylations with acetic anhydride, and therefore, that C_{hydro,p-SDS}(BCA-Hexₙ)˜3 C_{hydro,p-SDS} (BCA-Acₙ).

Intramolecular destabilization due to additional exposed hydrophobic surface area were determined as follows. The increase in the exposed hydrophobic area on the surface of the acylated BCA relative to BCA should also decrease the stability of the folded protein. The increase in exposed surface area increases the order of the water surrounding the protein, thereby decreasing the entropy in the folded state. The increase in ordered water molecules around hydrophobic residues relative to BCA should be greatest in the folded state; in the denatured state, there is a large exposed surface, and the change due to the chemical modification of lysine residues should be minimal. The ΔΔG^{o}_{folding} between acylated BCA and BCA is, then, primarily due to a destabilization of the ground state; this destabilization should also affect ΔΔG^{‡} because the transition state should be less affected than the folded state. (The configurational entropy of the modified side chains in the native and transition states may also contribute to the stability of the derivatives. The relative configurational entropy in the ground and the transition state could also increase the rate of denaturation of modified BCA relative to native BCA.)

The free energy required to transfer a hydrocarbon from the pure hydrocarbon phase to water is a linear function of the surface area of the chain. It has demonstrated that a change in hydrophobic surface area on a protein contributes 12-28 cal mol⁻¹ Å⁻² to ΔG⁰_{folding}. Using this stability scale as justification, it can be assumed that the difference in free energy of folding between acylated BCA and BCA (ΔΔG^{o}_{folding}), and also the destabilization of the folded state relative to the transition state (ΔΔG^{‡}), is linear with the number of acylations (Eq. 7):

ΔΔG^{‡}_{hydro,p}=C_{hydro,p} n (7),

where C_{hydro,p} is a constant of proportionality that differs between charge ladders and should be proportional to size of the surface area of the acylating reagent used. Because hexanoyl groups are ˜3 times the surface area of acetyl group, C_{hydro,p}(BCA-Hexₙ)=3 C_{hydro,p}(BCA-Acₙ).

A surface area calculation was also performed to determine the change in surface area between the conversion of BCA to BCA-Ac₁₈ and BCA to BCA-Hex₁₈. It was calculated that the reaction with acetic anhydride changed the surface area of BCA by 400 Å² and the reaction with hexanoic anhydride changed the surface area of BCA by 1180 Å². Since this change is a factor of 2.9, it can be concluded that this estimate that each modification with hexanoic anhydride adds three times the amount of surface area than modification with acetic anhydride is justified.

The changes to hydrophobicity in the model are treated as follows. Because ΔΔG^{‡}_{hydro,p-SDS} and ΔΔG^{‡}_{hydro,p} have the same functional form within this model, the same dependence on the number of modifications, and the same dependence on the identity of the anhydride (3 C_{hydro}(Ac)=C_{hydro}(Hex)), the intermolecular and intramolecular effects of changes in hydrophobicity cannot be distinguished. The cumulative ΔΔG^{‡}_{hydro} (Eq. 8) can be measured, however. Here, C_{hydro} is a proportionality constant and is equal to C_{hydro,p-SDS}+C_{hydro,p}:

ΔΔG^{‡}_{hydro}=ΔΔG^{‡}_{hydro,p-SDS}+ΔΔG^{‡}_{hydro,p}=C_{hydro}n (8).

Combination of electrostatic and hydrophobic terms into a single equation. By substituting Eqs. 3, 4, and 8 into Eq. 2, the activation energy of each rung of the charge ladder can be expressed as a function of the number of acylations, n (Eq. 9):

$$Δ {G}_{BCA - Xn}^{‡} = \begin{matrix}Δ {G}_{BCA}^{‡} + \frac{{me}_{c}^{2} Δ Zn}{4 {πɛ}_{0} {ɛ}_{w} d ( 1 + κ d )} - \\ \frac{{e}_{c}^{2}}{8 {πɛ}_{0} {ɛ}_{p}} ( \begin{matrix}\frac{1}{R ( 1 + κ R )} - \\ \frac{1}{{R}_{TS} ( 1 + κ {R}_{TS} )}\end{matrix} ) \times \\ ( - 2 {Z}_{0} Δ Zn + {n}^{2} ) - {C}_{hydro} n .\end{matrix} \qquad ( 9 )$$

The nonlinear term in Eq. 9 depends on intramolecular electrostatic repulsion. Since the data in FIG. 8A are nonlinear, it can be concluded that the intramolecular electrostatic repulsion is an important factor in the denaturation of proteins, especially when the net charge on the protein becomes large (>10 e_{c} for BCA). Since this repulsion depends only on parameters of the protein (and not the SDS molecules), it may play a role in protein stability and denaturation with other denaturants.

There are four unknown parameters (m, the number of molecules of SDS bound to the protein in the transition state, R_{TS}, the radius of the transition state, d, the distance between the SDS molecules and the protein in the transition state, and C_{hydro}, a constant representing the sum of the hydrophobic interactions) in Eq. 9. Those parameters using this model and the data in FIG. 8A can therefore be estimated to see if the calculated values for these parameters seem physically reasonable.

Analysis of the relative rates of denaturation of different rungs to the proposed model. The model predicts that a second-order polynomial describes the rate of denaturation as a function of the number of acylations. The data in FIG. 8A is fit to a second-order polynomial (a+bn+cn²), where n is the number of acylations. The fits of the two charge ladders were constrained to obtain the best fit for both ladders, with a and c constrained to the same value for both ladders because they describe the electrostatic terms (which were assumed to be invariant). FIG. 8A shows the fits. (If the data for BCA-Acₙ and BCA-Hexₙ are fit independently, the values for a and c for each data set are within error of each other.) The coefficients a and c are independent of the kind of acylation because they do not depend on the hydrophobicity of the reagent; they depend only on the electrostatic interactions. As a result, the difference in activation energies between acetyl and hexanoyl ladders (Eq. 10) should be linear (FIG. 8A):

ΔΔG^{‡}=ΔG^{‡}(BCA-Acₙ)−ΔG^{‡}(BCA-Hexₙ) (10).

### The slope (−0.17 kcal/mol of protein) is equal to ΔC_{hydro}, where ΔC_{hydro}=C_{hydro}(Ac)-C_{hydro}(Hex)=C_{hydro}(Ac)−3C_{hydro}(Ac)=−2 C_{hydro}(Ac). Therefore, C_{hydro}(Ac)=0.085 kcal/mol and C_{hydro}(Hex)=0.26 kcal/mol per acylation.

It was found that ΔG^{‡} for BCA (i.e., the a coefficient) was 14±1 kcal/mol; this value will be directly affected by the estimate that νκ_{TST}=10⁶ s⁻¹. If the value of νκ_{TST} is underestimated, the actual ΔG^{‡} for BCA will be lower. The c coefficient, due only to intramolecular electrostatic destabilization, was −0.023±0.001 kcal/mol of protein. (The negative sign on the c coefficient indicates that the intramolecular electrostatic repulsion decreases ΔG^{‡}. The negative sign is expected because this repulsion should destabilize the folded state of the protein and decrease the magnitude of the activation energy of denaturation.) Using the model, and assuming a radius of BCA of 2 nm, R_{TS} was calculated to be 2.1 nm. This radius is 5% larger than that of the folded protein, and the protein may remain relatively compact in the transition state.

The b coefficient for BCA-Acₙ was 0.50±0.01 kcal/mol of protein; the b coefficient for BCA-Hexn was 0.33±0.01 kcal/mol of protein. This parameter has four components (see Eq. 9): i), electrostatic repulsion between the protein and SDS molecules, ii), the linear portion of the intramolecular electrostatic term, iii), hydrophobic interaction between protein and SDS, and iv), destabilization of the protein due to exposed hydrophobic surface area. From the calculations above, C_{hydro}(acetyl)=0.085 kcal/mol and C_{hydro}(hexanoyl)=0.26 kcal/mol, and R_{TS}=2.1 nm. There are two remaining unknown parameters, d and m, but only one equation to constrain them. However, reasonable assumptions can be made about one of these parameters and the corresponding value for the other parameter can be determined to see if it is reasonable. If d is assumed to be the radius of the protein in the transition state (2.1 nm), a value for m of ˜7 molecules of SDS bound in the transition state can be determined. If m is ˜10 molecules of SDS, a value for d of 2.8 nm can be determined.

Here, d should not be much larger than the Debye length (3 nm in the buffer) or the interactions should be heavily screened. With the assumptions made in this highly simplified model, it can be concluded that there are ˜10 molecules of SDS bound in the transition state. Since this number is ˜1 order of magnitude lower than the ˜130 molecules of SDS bound to the protein when completely denatured, it can be concluded that there are a small number of SDS molecules that interact with the protein and cause changes to the conformation of the protein. The rest of the molecules of SDS thus bind to the denaturing protein in later, nonrate determining steps.

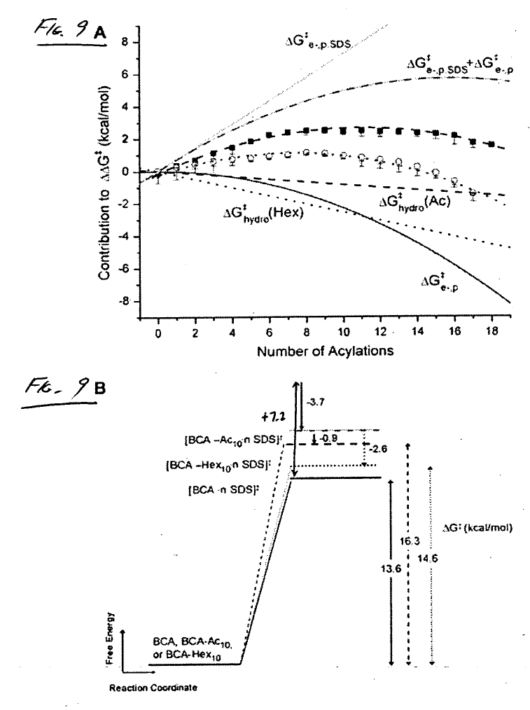

An interpretation of the results of the fitting is the following. FIG. 9 shows a schematic diagram of how the four effects in the model affect the height of the activation barrier. FIG. 9A shows contributions to ΔΔG^{‡} from ΔG^{‡}_{e-,p-SDS}, ΔG^{‡}_{e-,p}, and ΔG^{‡}_{hydro}. The sum of the electrostatic contributions is marked as the dashed line. The data for ΔΔG^{‡}—the sum of the four components—are shown for BCA-Acₙ (squares) and BCA-Hexₙ (circles). The fits to the data (dashed line, BCA-Acₙ; dotted line, BCA-Hexₙ) are those given by Eq. 9. FIG. 9B is an example of how the energy of the transition state changes relative to that of the ground state with 10 modifications. The folded states of BCA, BCA-Ac₁₀, and BCA-Hex₁₀ are scaled to the same energy. The arrows indicate how each of the factors changes the relative position of the transition state. The dashed line shows the effects of just the electrostatic terms on the energy of the transition state (i.e., if 10 lysine groups were neutralized with no corresponding change in hydrophobicity).

The figure also shows a plot of the contributions of ΔG^{‡}_{e-,p-SDS}, ΔG^{‡}_{e-,p}, and ΔG^{‡}_{hydro} to ΔΔG^{‡} between BCA and each rung of the charge ladder. For both BCA-Acₙ and BCA-Hexₙ, net electrostatics contribute more to ΔG^{‡} than hydrophobicity. The contribution to ΔΔG^{‡} due to the changes in net charge of the protein is shown as the dotted line. At low values of n, the ΔG^{‡}_{e-,p} term dominates and the modified proteins have a higher activation energy than the native BCA. At high values of n, the contributions of ΔG^{‡}_{e-,p-SDS} and ΔG^{‡}_{e-,p} largely offset each other. For BCA-Hexₙ, the effects of changes in hydrophobicity (dotted line) are nearly the same in magnitude as the effects of changes in electrostatics.

The data are consistent with the model presented. The assumptions discussed earlier are simplifications of a complex biochemical system, and the simplistic model can only begin to identify free energies that may be important in determining how the stability of a protein is changed by chemical modifications to that protein and how surfactants denature proteins. In conclusion, hydrophobic charge ladders are a useful tool for determining the relative importance of charge and hydrophobicity in the denaturation of proteins with SDS. Charge ladders provide data in which charge and hydrophobicity vary independently. These data allow quantitative estimations of the relative importance of electrostatics and hydrophobicity in the rate of denaturation of BCA (and other proteins) with SDS. In particular, the study with acetyl and hexanoyl charge ladders of BCA indicates that both charge and hydrophobicity affect the rate of denaturation of BCA with SDS. It can be concluded that the effects of charge on denaturation with SDS are ˜5-fold larger than the effects of hydrophobicity for BCA-Acₙ and of similar size for BCA-Hexₙ.

To account for the curvature in the data of rate of denaturation versus number of acylations, a nonlinear term must be included that describes intramolecular electrostatic repulsion. The functional form of the model described in this study fits well to the data for BCA-Acₙ and BCA-Hexₙ; it can be concluded that the four terms included in this model—inter- and intramolecular electrostatic repulsion, and inter- and intramolecular hydrophobic interactions—give a plausible description of the major factors in determining the change in the rate of denaturation with acylation. These results suggest that removing small amounts of negative charge (˜1-10 e_{c}) from the surface of a protein may stabilize that protein to denaturation with SDS, but that removing large amounts of charge (>15 e_{c}) will destabilize the protein. There is, therefore, an optimum amount of surface charge to make a protein stable to SDS denaturation and, at least for one protein (BCA), this ideal charge is different from that of the native protein. The strength of using charge ladders is that the effects are averaged over multiple species (regioisomers). The fact that the denaturation of the set of them represented by each rung of the ladder can be described with a simple, intuitive model, and a common set of numerical constants, implies that the rate of denaturation is dominated by global (nonlocal) effects.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. An article, comprising:
an enzyme, having a plurality of lysine residues, in which at least about 50% of the lysine resides of the enzyme have been acetylated.

2. The article of claim 1, wherein at least about 75% of the lysine residues of the enzyme have been acetylated.

3. The article of claim 2, wherein at least about 85% of the lysine residues of the enzyme have been acetylated.

4. The article of claim 3, wherein at least about 95% of the lysine residues of the enzyme have been acetylated.

5. The article of claim 1, wherein substantially all of the lysine residues of the enzyme have been acetylated.

6. The article of claim 1, wherein the enzyme has at least 5 lysine residues.

7. A method, comprising:
providing an enzyme able to catalyze a substrate; and
reacting a lysine residue of the enzyme with an acetylating agent to produce an acetylated enzyme such that the acetylated enzyme retains a specific activity, with respect to the substrate of the enzyme, of at least about 75% relative to the enzyme prior to the reacting step.

8. (canceled)

9. The method of claim 7, wherein the acetylated enzyme retains a specific activity of at least about 85%.

10. The method of claim 9, wherein the acetylated enzyme retains a specific activity of at least about 90%.

11. The method of claim 10, wherein the acetylated enzyme retains a specific activity of at least about 95%.

12. The method of claim 7, wherein the enzyme has at least 5 lysine residues.

13. The method of claim 7, comprising reacting at least about 50% of the lysine resides of the enzyme.

14-21. (canceled)

22. A method, comprising:
providing an enzyme able to catalyze a substrate; and
reacting a lysine residue of the enzyme with an acetylating agent to produce an acetylated enzyme such that the acetylated enzyme, when exposed to sodium dodecyl sulfate at a concentration of at least about 2.5 mM in Tris-Gly buffer, denatures with a time constant of less than about 175 h.

23-25. (canceled)

26. An article, comprising:
an acetylated enzyme that, when exposed to sodium dodecyl sulfate at a concentration of at least about 2.5 mM in Tris-Gly buffer, denatures with a time constant of less than about 175 h.

27-41. (canceled)

42. A method, comprising:
promoting use, in a detergent, of an enzyme in which a plurality of lysine residues within the enzyme have been reacted with an anhydride.

43-49. (canceled)
